(19) United States
(12) Patent Application Publication
(10) Pub. No.: US 2023/0270961 A1
(43) Pub. Date: Aug. 31, 2023

(54) LUNG-PROTECTIVE VENTILATION

(71) Applicant: Covidien LP, Mansfield, MA (US)

(72) Inventors: Kun LI, San Diego, CA (US); Nancy F. DONG, San Marcos, CA (US); Gabriel SANCHEZ, Valley Center, CA (US); Rhomere S. JIMENEZ, Chula Vista, CA (US)

(73) Assignee: Covidien LP, Mansfield, MA (US)

(21) Appl. No.: 18/175,210

(22) Filed: Feb. 27, 2023

Related U.S. Application Data
(60) Provisional application No. 63/268,659, filed on Feb. 28, 2022.

(51) Int. Cl. A61M 16/00; A61M 16/20

(52) U.S. Cl. CPC A61M 16/0051; A61M 16/204; A61M 2205/502; A61M 2016/0027; A61M 2205/583; A61M 2230/40

(57) ABSTRACT

Systems and methods for lung-protective ventilation are disclosed. In examples, volume-targeted, pressure-controlled ventilation may deliver mandatory breaths to a patient without a rise time setting. Inputs into the ventilation may include a peak inspiratory flow value (Qₚₑₐₖ) and a target tidal volume (V_{T,set}). Respiratory parameters of the patient may be determined based on test breaths. The inputs and the respiratory parameters may be used to calculate a target inspiratory pressure (Pᵢ) and target rise time constant (τ). Breaths may then be delivered based on the calculated target inspiratory pressure and target rise time constant. Mechanical power delivered to the patient may also be monitored as an additional measure for patient lung protection.

### CROSS-REFERENCE TO RELATED APPLICATIONS

This application claims the benefit of U.S. Provisional Application No. 63/268,659 filed Feb. 28, 2022, entitled “Lung-Protective Ventilation,” which is incorporated herein by reference in its entirety.

### INTRODUCTION

Medical ventilator systems have long been used to provide ventilatory and supplemental oxygen support to patients. These ventilators typically comprise a connection for pressurized gas (air, oxygen) that is delivered to the patient through a conduit or tubing. As each patient may require a different ventilation strategy, modern ventilators may be customized for the particular needs of an individual patient. For example, several different ventilator modes or settings have been created to provide better ventilation for patients in different scenarios, such as mandatory ventilation modes, spontaneous ventilation modes, and assist-control ventilation modes. Ventilators monitor a variety of patient parameters and are well equipped to provide reports and other information regarding a patient's condition.

It is with respect to this general technical environment that aspects of the present technology disclosed herein have been contemplated. Furthermore, although a general environment is discussed, it should be understood that the examples described herein should not be limited to the general environment identified herein.

### SUMMARY

This summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

Among other things, aspects of the present disclosure include systems and methods for lung-protective ventilation. In an aspect, the technology relates to a method for lung-protective ventilation. The method includes receiving, as input to the ventilator, a target tidal volume and a peak inspiratory flow; delivering an initial breath based on initial ventilation settings; determining respiratory parameters, based on the delivered initial breath; calculating a target rise time constant and a target inspiratory pressure, based at least on the received target tidal volume, the peak inspiratory flow, and the determined respiratory parameters; and delivering a target breath according to the target rise time constant and target inspiratory pressure.

In an example, the initial ventilation settings include an initial rise time constant, an initial inspiratory pressure, and a peak inspiratory pressure. In another example, the method further includes measuring an actual tidal volume delivered during the target breath; adjusting the target inspiratory pressure to an adjusted inspiratory pressure, based on the actual tidal volume; and delivering a third breath according to the target rise time and the adjusted inspiratory pressure. In a further example, adjusting the target inspiratory pressure is further based on a difference between the actual tidal volume and the target tidal volume. In still another example, rise time is not available as a setting capable of being adjusted by a clinician. In yet another example, the method further includes calculating a mechanical power value based on the target rise time constant and the target inspiratory pressure. In still yet another example, the method further includes displaying the calculated mechanical power and indicia indicating a relationship between the calculated mechanical power value and a mechanical power threshold.

In another example, calculating the target rise time constant and the target inspiratory pressure is based on a least squares optimization technique. In still another example, the target breath is delivered according to a flow profile based on the target rise time constant and the target inspiratory pressure, wherein a flow of breathing gases delivered to a patient is variable based on the flow profile. In yet another example, the method further includes displaying at least one of: the target rise time; the target inspiratory pressure; or a mechanical power value calculated based on the target rise time and the target inspiratory pressure.

In another aspect, the technology relates to a method for lung-protective ventilation. The method includes receiving a target tidal volume and peak inspiratory flow as inputs to the ventilator; determining respiratory parameters; calculating a rise time constant and a first target inspiratory pressure based at least on the respiratory parameters and the target tidal volume; delivering a first breath according to the rise time constant and the first target inspiratory pressure; measuring an actual tidal volume delivered during the first breath; determining that the actual tidal volume is outside of a volume range from the target tidal volume; adjusting the first target inspiratory pressure to a second target inspiratory pressure; and delivering a second breath, based on the rise time constant and the second target inspiratory pressure.

In an example, the actual tidal volume is below the volume range, and the second target inspiratory pressure is greater than the first target inspiratory pressure. In another example, the actual tidal volume is higher than the volume range, and the second target inspiratory pressure is lower than the first target inspiratory pressure. In still another example, calculating the rise time constant and the first target inspiratory pressure is further based on a peak inspiratory pressure. In yet another example, the method further includes receiving an initial rise time constant and an initial inspiratory pressure; and delivering an initial breath, based on the initial rise time constant and the initial inspiratory pressure, wherein respiratory parameters are based on the initial breath.

In another aspect, the technology relates to a ventilator for lung-protective ventilation. The ventilator includes a flow valve; a flow sensor; a user interface; a processor; and memory storing instructions that, when executed by the processor, cause the ventilator to perform a set of operations. The operations include receiving, at the user interface, a target tidal volume and a peak inspiratory flow; delivering a first breath through the flow valve; determining respiratory parameters, based on the first breath; calculating a target rise time constant and a first target inspiratory pressure, based at least on the determined respiratory parameters, the target tidal volume, and the peak inspiratory flow; delivering a second breath, through the flow valve according to the target rise time constant and the first target inspiratory pressure; calculating, based on measurements from the flow sensor, an actual tidal volume delivered during the second breath; adjusting the first target inspiratory pressure to a second target inspiratory pressure, based on a difference between the actual tidal volume and the target tidal volume; and delivering a third breath, through the flow valve according to the target rise time and the second target inspiratory pressure.

In an example, the operations further include displaying, in the user interface, the actual tidal volume and at least one of: the target rise time constant, the first target inspiratory pressure, or the second target inspiratory pressure. In another example, the first breath and the second breath are delivered according to a volume-targeted, pressure-controlled, mandatory breath mode. In still another example, the ventilator further includes a pressure sensor, wherein the flow valve is controlled based at least on a measurement from the pressure sensor while delivering the second breath and the third breath. In yet another example, the operations further include calculating a mechanical power for the third breath, based at least on the target rise time and the second target inspiratory pressure; and alarming based on the calculated mechanical power.

In another aspect, the technology relates to a method for lung-protective ventilation. The method includes delivering, by a mechanical ventilator, an inspiratory breath according to a rise time constant and a target inspiratory pressure; measuring, by the mechanical ventilator, at least one or pressure values or flow values associated with the delivered breath; based on the measured values, calculating a tidal volume delivered by the breath; dynamically adjusting at least one of the rise time constant or the target inspiratory pressure for a subsequent breath to drive the delivered tidal volume toward a target tidal volume, wherein dynamically adjusting comprises: determining that the delivered tidal volume differs from the target tidal volume; based on determining that the delivered tidal volume differs from the target tidal volume, adjusting at least one of: the rise time constant to an adjusted rise time constant; or the target inspiratory pressure to an adjusted target inspiratory pressure; and delivering the subsequent breath according to at least one of the adjusted rise time constant or the adjusted target inspiratory pressure.

It is to be understood that both the foregoing general description and the following Detailed Description are explanatory and are intended to provide further aspects and examples of the disclosure as claimed.

### BRIEF DESCRIPTION OF THE DRAWINGS

The following drawing figures, which form a part of this application, are illustrative of aspects of systems and methods described below and are not meant to limit the scope of the disclosure in any manner, which scope shall be based on the claims.

FIG. 1 is a diagram illustrating an example of a medical ventilator connected to a human patient.

FIG. 2 is a block-diagram illustrating an example of a ventilator system.

FIG. 3 is a flowchart illustrating an example method for lung-protective ventilation.

FIG. 4 is a flowchart illustrating another example method for lung-protective ventilation.

FIG. 5 is a flowchart illustrating another example method for lung-protective ventilation.

FIG. 6 is a flowchart illustrating another example method for lung-protective ventilation.

FIG. 7 is a flowchart illustrating an example method for monitoring mechanical power.

FIG. 8 shows an example graphical user interface for lung-protective ventilation.

While examples of the disclosure are amenable to various modifications and alternative forms, specific aspects have been shown by way of example in the drawings and are described in detail below. The intention is not to limit the scope of the disclosure to the particular aspects described. On the contrary, the disclosure is intended to cover all modifications, equivalents, and alternatives falling within the scope of the disclosure and the appended claims.

### DETAILED DESCRIPTION

As discussed briefly above, medical ventilators are used to provide breathing gases to patients who are otherwise unable to breathe sufficiently. In modern medical facilities, pressurized air and oxygen sources are often available from wall outlets, tanks, or other sources of pressurized gases. Accordingly, ventilators may provide pressure regulating valves (or regulators) connected to centralized sources of pressurized air and pressurized oxygen. The regulating valves function to regulate flow so that respiratory gases having a desired concentration are supplied to the patient at desired pressures and flow rates. Further, as each patient may require a different ventilation strategy, modern ventilators may be customized for the particular needs of an individual patient.

For the purposes of this disclosure, a “breath” refers to a single cycle of inspiration and exhalation delivered with the assistance of a ventilator. The term “breath type” refers to some specific definition or set of rules dictating how the pressure and flow of respiratory gas are controlled by the ventilator during a breath. For example, breath types may be mandatory mode breath types where the initiation and termination of the breath is made by the ventilator, or spontaneous mode breath types where the breath is initiated (and in some cases also terminated) by the patient. Examples of spontaneous breath types include proportional assist (PA) breath type, volume support (VS) breath type, pressure support (PS) breath type, etc. Examples of mandatory breath types include a volume control breath type, a pressure control breath type, volume-targeted pressure control breath type etc.

A ventilation “mode,” on the other hand, is a set of rules controlling how multiple subsequent breaths should be delivered. A simple mandatory mode of ventilation is to deliver one breath of a specified mandatory breath type at a clinician-selected respiratory rate, f (e.g., one breath every 6 seconds). A single ventilation mode may mix different breath types, delivering a first breath type and then in a subsequent breath delivering a second, different breath type, according to the rules of the mode. Typically, ventilators will continue to provide breaths of the specified breath type(s) as dictated by the rules defining the mode, until the mode is changed by a clinician.

Regarding mandatory mode breath types, conventional ventilation may include at least three modalities: (1) volume-controlled ventilation (VCV), (2) pressure-controlled ventilation (PCV), and (3) pressure-regulated volume control ventilation (PRVC). In any of these modes, ventilator-induced lung injury (VILI) may result if ventilation settings are not adjusted for an individual patient over the course of treatment, or from patient-to-patient. For example, the occurrence and/or severity of VILI may depend on the ventilation settings such as tidal volume (V_{T}), inspiratory pressure (Pᵢ), flow rate (Q or \dot{V}), respirate rate (f), and/or positive end-expiratory pressure (PEEP).

To reduce or prevent VILI, a lung-protective ventilation strategy may be employed. In particular, a lung-protective strategy may be desirable for patients with an acute lung injury and/or acute respiratory distress syndrome (ARDS). Lung-protective ventilation may limit inspiratory pressure (e.g., <40 cmH₂O, <50 cmH₂O, <100 cmH₂O, etc.), tidal volume constant (e.g., ≤6 mL/kg, ≤7 mL/kg, ≤8 mL/kg, ≤10 mL/kg, etc.), and/or PEEP. For volume-targeted ventilation, the tidal volume (V_{T}) of gas delivered to the patient is targeted and for pressure-targeted ventilation, the inspiratory pressure (Pᵢ) is targeted.

Different modes or breath types may be set up to deliver the same tidal volume target in different ways. Volume-controlled ventilation (VCV) mode may control the inspiratory flow rate with a clinician-set flow profile, such as constant flow, decelerating flow, sinusoidal flow, etc., while the airway pressure is limited by a preset value. The control mechanisms of VCV, however, may be sensitive to airway leaks, which may compromise volume accuracy. To address airway leaks, hybrid modes may be used, such as volume-guarantee (VG) ventilation, pressure-regulated volume control (PRVC), or volume-control plus (VC+). Each of these hybrid modes use pressure control to achieve the same tidal volume target (V_{T}) as in VCV. These hybrid modes, which use pressure-controlled ventilation (PCV), differ from VCV by varying the inspiratory flow rate during inhalation. In PCV, the inspiratory flow rate may rise to a peak inspiratory flow value (Qₚₑₐₖ) near the beginning of an inhalation phase, then gradually decrease towards the end of the inhalation phase. The peak inspiratory flow value (Qₚₑₐₖ) may depend on patient respiratory system mechanics (e.g., compliance, resistance, elastance, etc.), as well as the ventilator settings such as tidal volume target (V_{T}) and rise time setting ( [symbol] P).

Excessive inspiratory flow rates during PCV may lead to increased likelihood of VILI. This is particularly true for preterm infants, as excessive inspiratory flow rates can cause severe damage to the lungs. The increased likelihood of VILI due to excessive inspiratory flow rates may be based on additional stress caused by pendelluft (e.g., the movement of gas between two regions of the lung, usually between regions of differing compliance or airway resistance). Although some ventilators have maximum flow rate settings associated with patient types (e.g., 200 L/min for an adult and 50 L/min for a neonate), reaching these maximum flow rate settings may cause VILI for some patients. Also associated with VILI is excessive mechanical power delivered to a patient. Thus, to reduce stress during PCV, the inspiratory flow rate and/or mechanical power may be limited or controlled.

One technique that may be used to control inspiratory flow rates is the rise time setting (FP). Some ventilators include a settable rise time setting in pressure-controlled ventilation and high flow ventilation. The rise time setting (or rise time %) specifies the speed at which the inspiratory pressure reaches a target, such as 95% of the target inspiratory pressure (PO. A higher rise time setting is associated with a higher peak inspiratory flow value (Qₚₑₐₖ) to achieve the target inspiratory pressure (Pᵢ) in a shorter period of time during an inhalation phase. A lower rise time setting is associated with a lower peak inspiratory flow value (Qₚₑₐₖ) to achieve the target inspiratory pressure (Pᵢ) over a longer period of time during an inhalation phase. Generally, a desirable rise time setting for gently breathing patients or for patients with high impedance (e.g., low compliance and high resistance) is less than or equal to the median value of the full setting range (such as 50% out of the full range of 1%-100%), which may be a default setting on a ventilator. This setting may provide a longer rise time and a lower Qₚₑₐₖ. In contrast, a desired rise time setting for more aggressively breathing patients or patients with low impedance (e.g., high compliance and low resistance) is greater than or equal to 50%. This setting may provide a shorter rise time and a higher Qₚₑₐₖ. Thus, the rise time setting may be used to control the peak inspiratory flow value (Qₚₑₐₖ) and reduce or prevent VILI.

The rise time setting, however, is often not adjusted to a value that is desirable or best-suited for the patient. In some situations, the rise time setting is untouched or maintained at a default setting (e.g., 50%). As described above, this default value is not best-suited for all patients. For example, gently breathing patients or patients with high impedance may benefit from rise time settings less than a default setting to reduce or prevent VILI. In other situations, the rise time setting is set to the maximum value with the intention to achieve a near-square pressure waveform, which can deliver a peak flow rate (Qₚₑₐₖ) that is far too high for the patient. Moreover, this result can occur without the user's knowledge of the peak flow rate (Qₚₑₐₖ) being delivered to the patient. Under certain clinical circumstances (such as stiff lungs or a small patient with a weak inspiratory drive), a rise time setting above the default may cause a transient pressure overshoot and premature transition to exhalation or pressure oscillations during inspiration.

Among other things, the systems and methods disclosed herein address these circumstances by providing a lung-protective ventilation mode without allowing a user to set or adjust a rise time setting. Instead, a peak inspiratory flow value (Qₚₑₐₖ) may be set and/or adjusted by a clinician. Additionally, a target tidal volume (V_{T,set}) may be set, but no rise time setting may be received from the user. This limitation on user input safeguards the delivered breath from targeting a rise time that is too fast or too slow. Rather than rely on the user to specify the rise time, the rise time follows from a determination of a lung-protective breath based on peak inspiratory flow and target tidal volume. The rise time adjusts dynamically based on the respiratory parameters of the patient and the desired ventilation, to avoid a static rise time setting that is too low or too high. The lung-protective ventilation mode may provide volume-targeted, pressure-controlled ventilation based on estimated respiratory system mechanics (e.g., resistance, compliance, etc.) and by calculating a target inspiratory pressure (Pᵢ) and dynamically calculating a target rise time constant (τ). Mechanical power delivered to the patient may also be monitored or used as an additional measure for patient lung protection. With these concepts in mind, several examples of lung-protective ventilation methods and systems are discussed below.

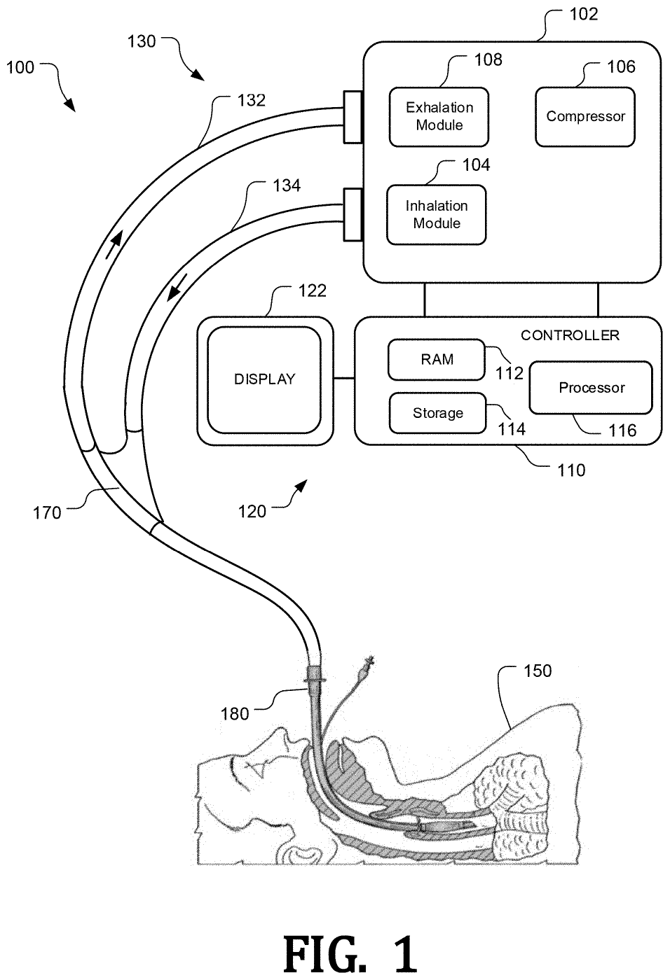

FIG. 1 is a diagram illustrating an example of a medical ventilator 100 connected to a human patient 150. The ventilator 100 provides positive pressure ventilation to the patient 150. Ventilator 100 includes a pneumatic system 102 (also referred to as a pressure generating system 102) for circulating breathing gases to and from patient 150 via the ventilation tubing system 130, which couples the patient to the pneumatic system via an invasive (e.g., endotracheal tube, as shown) or a non-invasive (e.g., nasal mask) patient interface.

Ventilation tubing system 130 may be a two-limb (shown) or a one-limb circuit for carrying gases to and from the patient 150. In a two-limb example, a fitting, typically referred to as a “wye-fitting” 170, may be provided to couple a patient interface 180 to an inhalation limb 134 and an exhalation limb 132 of the ventilation tubing system 130.

Pneumatic system 102 may have a variety of configurations. In the present example, system 102 includes an exhalation module 108 coupled with the exhalation limb 132 and an inhalation module 104 coupled with the inhalation limb 134. Compressor 106 or other source(s) of pressurized gases (e.g., air, oxygen, and/or helium) is coupled with inhalation module 104 to provide a gas source for ventilatory support via inhalation limb 134. The pneumatic system 102 may include a variety of other components, including mixing modules, valves, sensors, tubing, accumulators, filters, etc., which may be internal or external sensors to the ventilator (and may be communicatively coupled, or capable communicating, with the ventilator). For example, the pneumatic system 102 may include a flow valve to control the flow of breathing gases through the inhalation limb 134 and/or a pressure sensor to measure a pressure along the inhalation limb 134.

Controller 110 is operatively coupled with pneumatic system 102, signal measurement and acquisition systems, and an operator interface 120 that enables an operator to interact with the ventilator 100 (e.g., change ventilation settings, select operational modes, view monitored parameters, etc.). Controller 110 includes memory 112, one or more processors 116, storage 114, and/or other components of the type found in command and control computing devices. In the depicted example, operator interface 120 includes a display 122 that may be touch-sensitive and/or voice-activated, enabling the display 122 to serve both as an input and output device.

The memory 112 includes non-transitory, computer-readable storage media that stores software that is executed by the processor 116 and which controls the operation of the ventilator 100. In an example, the memory 112 includes one or more solid-state storage devices such as flash memory chips. In an alternative example, the memory 112 may be mass storage connected to the processor 116 through a mass storage controller (not shown) and a communications bus (not shown). Although the description of computer-readable media contained herein refers to a solid-state storage, the computer-readable storage media may be any available media that can be accessed by the processor 116. That is, computer-readable storage media includes non-transitory, volatile and non-volatile, removable and non-removable media implemented in any method or technology for storage of information such as computer-readable instructions, data structures, program modules or other data. For example, computer-readable storage media includes RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer. Communication between components of the ventilator system or between the ventilator system and other therapeutic equipment and/or remote monitoring systems may be conducted over a distributed network via wired or wireless means.

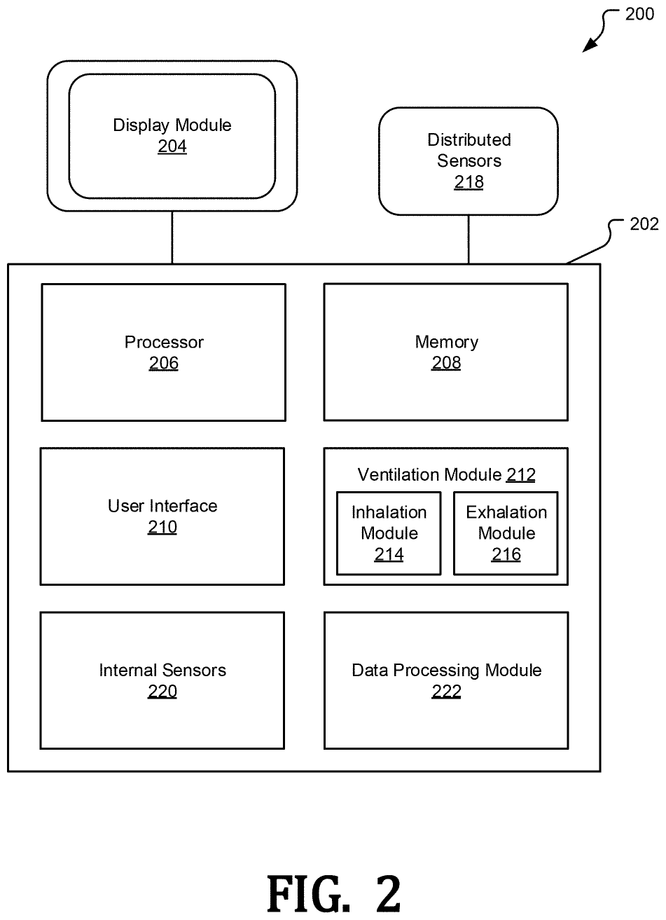

FIG. 2 is a block-diagram illustrating an example of a ventilator system 200. Ventilator system 200 includes ventilator 202 with various modules and components. That is, ventilator 202 may further include, among other things, hardware memory 208, one or more processors 206, user interface 210, and ventilation module 212 (which may further include an inhalation module 214 and an exhalation module 216). Memory 208 is defined as described above for ventilation module 212. Similarly, the one or more processors 206 are defined as described above for one or more processors 206.

The ventilator system 200 may also include a display module 204 communicatively coupled to ventilator 202. Display module 204 provides various input screens, for receiving input, and various display screens, for presenting useful information. Inputs may be received from a clinician. The display module 204 is configured to communicate with user interface 210 and may include a graphical user interface (GUI). The GUI may be an interactive display, e.g., a touch-sensitive screen or otherwise, and may provide various windows (i.e., visual areas) comprising elements for receiving user input and interface command operations and for displaying ventilatory information (e.g., ventilatory data, alerts, patient information, parameter settings, modes, waveforms, etc.). The elements may include controls, graphics, charts, tool bars, input fields, icons, etc. Alternatively, other suitable means of communication with the ventilator 202 may be provided, for instance by a wheel, keyboard, mouse, or other suitable interactive device. Thus, user interface 210 may accept commands and input through display module 204. Display module 204 may also provide useful information in the form of various ventilatory data regarding the physical condition of a patient and/or a prescribed respiratory treatment. The useful information may be derived by the ventilator 202, based on data collected and processed by a data processing module 222. The data processing module 222 is operative to determine a ventilation settings (otherwise referred to as ventilatory settings, or ventilator settings, or ventilation settings) associated with lung-protective ventilation, and to generate information for display.

Ventilation module 212 oversees ventilation of a patient according to ventilation settings. Ventilation settings may include any appropriate input for configuring the ventilator to deliver breathable gases to a particular patient, including measurements and settings associated with inhalation and exhalation flow of the breathing circuit. Ventilation settings may be entered, e.g., by a clinician based on a prescribed treatment protocol for the particular patient, or automatically generated by the ventilator, e.g., based on attributes (i.e., age, diagnosis, ideal body weight, predicted body weight, gender, ethnicity, etc.) of the particular patient according to any appropriate standard protocol or otherwise, such as may be determined in association with lung-protective ventilation. Ventilation settings may include inhalation flow, frequency of delivered breaths (e.g., respiratory rate f), tidal volume (V_{T}), PEEP level, and others.

In an embodiment, the ventilation module 212 includes an inhalation module 214 configured to deliver gases to the patient and an exhalation module 216 configured to receive exhalation gases from the patient. As described herein, inhalation module 214 may correspond to the inhalation module 104, and is coupled to source(s) of pressurized gases (e.g., air, oxygen, and/or helium), to deliver gases to the patient. As further described herein, exhalation module 216 may correspond to the exhalation module 108, and is coupled to gases exiting the breathing circuit.

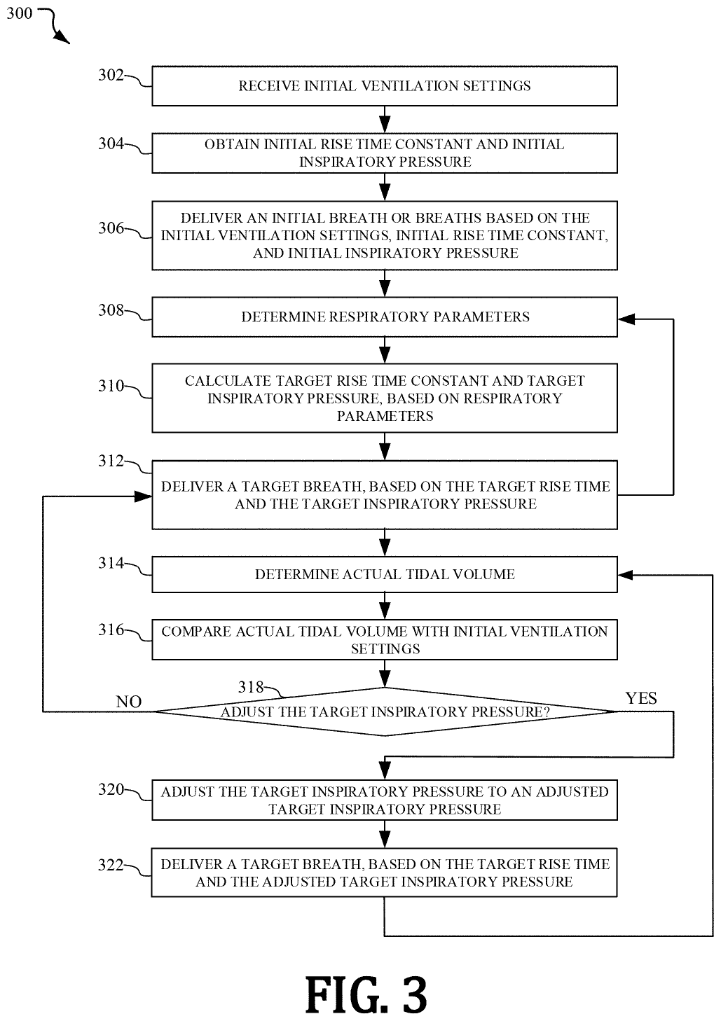

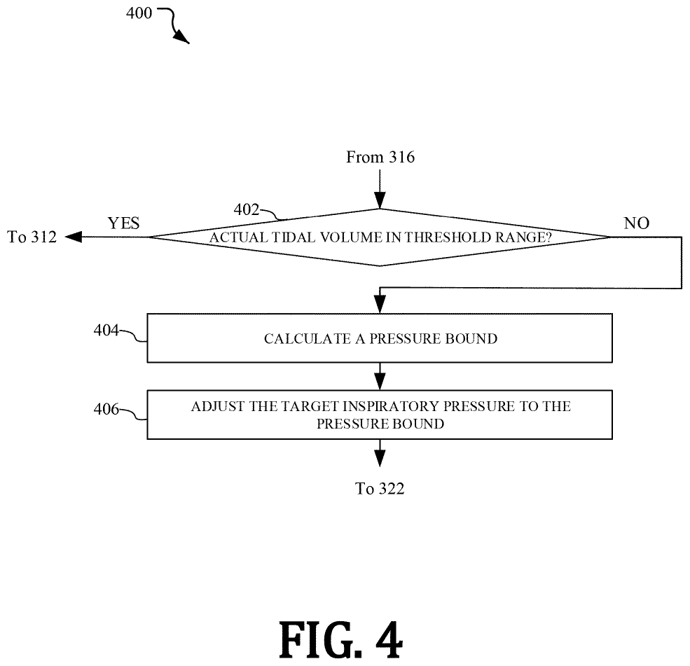

FIGS. 3 and 4 show example methods according to the disclosed technology. The example methods include operations that may be implemented or performed by the systems and devices disclosed herein. For example, ventilator 100 depicted in FIG. 1 and/or ventilator system 200 depicted in FIG. 2 may perform the operations described in the methods. In addition, instructions for performing the operations of the methods disclosed herein may be stored in a memory of the ventilator or ventilator system (e.g., system memory 112, 208 described in FIGS. 1 and 2) and executed by one or more processors of the respective systems.

FIG. 3 is a flowchart illustrating an example method 300 for lung-protective ventilation. At operation 302, initial ventilation settings are loaded, such as being received from a user or from preset or default values. Initial ventilation settings may include an inspiratory time (Tᵢ), a peak flow (Qₚₑₐₖ), and a target tidal volume (V_{T,set}). Other initial ventilation settings may include a peak pressure (Pₚₑₐₖ), a predicted body weight (PBW), a respiratory rate (f), an oxygen percentage, and a PEEP level. The initial ventilation settings may be received at a user interface or graphical user interface of a ventilator. One or more settings of the initial ventilation settings may be a preset value (e.g., a default value), calculated, or otherwise determined. For example, the target tidal volume (V_{T,set}) may be determined based on a PBW of a patient and a tidal volume ratio based on the body weight (e.g., 6 mL/kg, 7 mL/kg, 8 mL/kg, etc.).

At operation 304, an initial rise time constant (τₛₑₜ) and an initial target inspiratory pressure (P_{i,set}) are obtained or accessed. The initial rise time constant (τₛₑₜ) and an initial target inspiratory pressure (P_{i,set}) may be referred to collectively as initial protective parameters. The initial protective parameters may be based on default or derived values where no clinician input is requested or allowed. For example, the initial protective parameters may be a preset value (e.g., a default value), calculated, or otherwise determined. The initial rise time constant (τₛₑₜ) may be based on an inspiratory time (Tᵢ), such as Tᵢ/2, Tᵢ/3, Tᵢ/4, Tᵢ/5, or any value between zero and Tᵢ The initial target inspiratory pressure (P_{i,set}) may be based on one or more of the initial ventilation settings and/or the initial rise time constant (τₛₑₜ). For example, the initial rise time constant (τₛₑₜ) may be obtained or calculated (e.g., determined by the ventilator based on a received inspiratory time) along with one of the peak flow, peak pressure, and/or set tidal volume to estimate a value for the initial target inspiratory pressure (P_{i,set}) (e.g., using one or more of the equations described below).

In addition to, or alternatively to, the initial protective parameters including the initial rise time constant (τₛₑₜ) and the initial target inspiratory pressure (P_{i,set}), the initial protective parameters may include an initial mechanical power (MPₛₑₜ) to be delivered by the ventilator during an inhalation phase or a maximum mechanical power to be delivered during an inhalation phase. For example, the initial protective parameters may include one of the following sets of parameters: (1) τₛₑₜ and P_{i,set}; (2) τₛₑₜ and MPₛₑₜ; (3) P_{i,set} and MPₛₑₜ; or (4) τₛₑₜ, P_{i,set}, and MPₛₑₜ. The initial mechanical power may be received at a user interface or graphical user interface of a ventilator, may be preset, calculated, or otherwise determined. In an example, the initial mechanical power may be based on one or more of the initial ventilation settings, the initial rise time constant (τₛₑₜ), and/or the initial target inspiratory pressure (P_{i,set}).

At operation 306, the ventilator delivers an initial breath or breaths. The initial breath(s) may be based on the initial ventilation settings and the initial protective parameters obtained at operations 302 and 304, respectively. The initial breath(s) may be one or more test breaths delivered to a patient to determine respiratory parameters for the patient. For example, a patient may be monitored during one, two, three, or more initial breaths (e.g., delivered based on the initial ventilation settings and the initial protective parameters) to determine respiratory parameters of the patient.

At operation 308, respiratory parameters are determined. The respiratory parameters may be determined based on the initial breath(s) delivered at operation 306. The respiratory parameters may include the patient respiratory mechanics, and may include a resistance (R_{aw}), a compliance (C_{RS}), an elastance (E_{RS}), and/or a respiratory system time constant (τ_{RS}). One or more of the respiratory parameters may be determined using a pause maneuver (e.g., inspiratory pause maneuver, expiratory pause maneuver, or other pause maneuver).

At operation 310, a target rise time constant (τ) and a target inspiratory pressure (Pᵢ) are calculated. The target rise time constant (τ) and the target inspiratory pressure (Pᵢ) may be calculated based on the respiratory parameters determined at operation 308. In particular, the target rise time constant (τ) and the target inspiratory pressure (Pᵢ) may be calculated as two unknown variables common to two equations. The two equations used to determine the target rise time constant (τ) and the target inspiratory pressure (Pᵢ) may be selected from (1) the tidal volume over an inspiratory phase (e.g., as shown in Eqn. 3, below), (2) the peak flow rate over the inspiratory phase (e.g., as shown in Eqn. 4, below), and (3) the mechanical power (e.g., as shown in Eqn. 5).

The pressure profile, P(t), delivered at a given time, t, may be calculated using two different equations: (1) an inspiratory pressure trajectory equation (Eqn. 1, below), and (2) the equation of motion (Eqn. 2, below).

$$P ( t ) = {P}_{i} \times ( 1 - {e}^{- \frac{t}{τ}} ) + PEEP , 0 \leq t \leq {T}_{i} \qquad ( 1 )$$

$$P ( t ) = Q ( t ) \times {R}_{a w} + {E}_{R S} \times {\int }_{0}^{{T}_{i}} Q ( t ) d t + PEEP \qquad ( 2 )$$

In the above equations and throughout the present disclosure, Tᵢ is the inspiratory time, Pᵢ is the target inspiratory pressure above set PEEP, τ is the target rise time constant of the inspiratory pressure trajectory, Q(t) is the flow rate at time t, R_{aw} is the resistance of the patient's airway, and E_{RS} is the elastance of the patient's respiratory system (E_{RS}=1/C_{RS}, where C_{RS} is the compliance of the patient's respiratory system). As described above, Tᵢ and PEEP may be set by a clinician in the initial ventilation settings at operation 302. R_{aw} and E_{RS} may be determined with the respiratory parameters at operation 308. The target inspiratory pressure (Pᵢ) and the target rise time constant (τ) are unknown and to be calculated at this operation 310.
Based on Eqn. 1 and 2, the tidal volume delivered to the patient and the peak flow rate during the inhalation phase can be described by the following equations respectively:

$${V}_{T} = {\int }_{0}^{{T}_{i}} Q ( t ) dt = \frac{{P}_{i}}{{R}_{a w}} \times \frac{{τ}_{R S}}{{τ}_{R S} - τ} \times [ {τ}_{R S} \times ( 1 - {e}^{( - \frac{{T}_{i}}{{τ}_{R S}} )} ) - τ \times ( 1 - {e}^{( - \frac{{T}_{i}}{τ} )} ) ] \qquad ( 3 )$$

$${Q}_{p e a k} = Q ( {t}_{p e a k} ) = \frac{{P}_{i}}{{R}_{a w}} \times \frac{{τ}_{R S}}{{τ}_{R S} - τ} \times [ {( \frac{τ}{{τ}_{R S}} )}^{\frac{τ}{{τ}_{R S} - τ}} - {( \frac{τ}{{τ}_{R S}} )}^{\frac{{τ}_{R S}}{{τ}_{R S} - τ}} ] \qquad ( 4 )$$

where τ_{RS}=R_{aw}/E_{RS}=R_{aw}*C_{RS} is the time constant of the respiratory system.

As described above, the tidal volume over an inspiratory phase (e.g., as shown in Eqn. 3) and the peak flow rate over the inspiratory phase (e.g., as shown in Eqn. 4) may be used to calculate the target rise time constant (τ) and the target inspiratory pressure (Pᵢ). Because the equations for tidal volume and peak flow rate are nonlinear, however, analytic solutions are not available. Instead, the solutions can be obtained through an optimization technique, such as constrained least squares optimization or other techniques for obtaining solutions to non-linear equations. The values of the target inspiratory pressure (Pᵢ) and the target rise time constant (τ) may be constrained during the optimization technique. For example, the target inspiratory pressure (Pᵢ) may be constrained between zero and a peak inspiratory pressure (Pₚₑₐₖ), as set by the clinician at operation 302, and the target rise time constant (τ) may be constrained between zero and the inspiratory time (Tᵢ), as set by the clinician at operation 302.

In a specific example, the initial ventilation settings received at operation 302 include a target tidal volume of V_{T,set}=1200 mL and an inspiratory time of Tᵢ=1.0 s. The initial rise time constant and the initial inspiratory pressure obtained at operation 304 may be set internally with the default values. After delivering an initial breath at operation 306 according to the initial ventilation settings, initial rise time constant, and initial inspiratory pressure, respiratory parameters may be determined at operation 308 as follows: R_{aw}=5 cmH₂O/(L/s), E_{RS}=1/0.06 cmH₂O/L, and τ_{RS}=R_{aw}/E_{RS}=0.3 s. Using a constrained Least-Squares optimization technique based on Eqn. 3 and Eqn. 4, described above at operation 310, the target rise time constant (τ) and the target inspiratory pressure (Pᵢ) may be calculated as τ=0.188 s and Pᵢ=21.9 cmH₂O.

Mechanical power (MP) may also be determined and used in conjunction with the present technology. MP is the energy delivered to a patient's respiratory system over time during mechanical ventilation. The MP for volume-targeted ventilation (e.g., a pressure-controlled ventilation with a volume target) is expressed in Eqn. 5, below:

$$M P = 0.098 \times f \times {V}_{T} \times PEEP + 0.098 \times f \times \frac{{P}_{i}^{2}}{{R}_{a w}} \times \frac{{τ}_{R S}}{{τ}_{R S} - τ} \times \{ {τ}_{R S} \times [ 1 - {e}^{( - \frac{{T}_{i}}{{τ}_{R S}} )} ] - τ \times [ 1 - {e}^{( - \frac{{T}_{i}}{τ} )} ] + \frac{τ}{2} \times [ 1 - {e}^{( - \frac{2 \times {T}_{i}}{τ} )} ] - \frac{τ \times {τ}_{R S}}{τ + {τ}_{R S}} \times [ 1 - {e}^{( - \frac{τ + {τ}_{R S}}{τ \times {τ}_{R S}} \times {T}_{i} )} ] \} \qquad ( 5 )$$

In an example, the mechanical power (e.g., Eqn. 5) is one of the two equations used to calculate a target inspiratory pressure and a target rise time constant. For instance, a set maximum or desired MP may be provided by clinician (e.g., a mechanical power received at operation 302 or operation 304 may be used as the value for MP). Alternatively, if the mechanical power is not one of the two equations used to calculate the target inspiratory pressure and a target rise time constant, the mechanical power may still be monitored. For example, after calculating the target inspiratory pressure and the target rise time constant using other equations, mechanical power can be calculated using Eqn. 5. The value calculated for the mechanical power may be displayed. Additionally or alternatively, an alarm may be issued if the mechanical power exceeds a threshold. The threshold may be based on one or more patient parameters or may be preset or set by a user.

When calculating the target rise time constant (τ) and the target inspiratory pressure (Pᵢ) at operation 310, a difference between the target inspiratory pressure (Pᵢ) and the initial inspiratory pressure may be evaluated. Because large changes in the delivered inspiratory pressure may result in VILI, the difference between the target inspiratory pressure (Pᵢ) and the initial inspiratory pressure (P_{i,set}) may be bounded or limited. For example, the inspiratory pressure difference (e.g., ΔPᵢ=abs(Pᵢ−P_{i,set})) may be limited to a specified threshold value (e.g., ΔP_{i,max}=5 cmH₂O, 10 cmH₂O, 20 cmH₂O, etc.). In another example, a maximum inspiratory pressure difference may be based on one or more of the initial ventilation settings, such as a percentage of the initial inspiratory pressure (e.g., 5%, 10%, 15%, 20%, etc. of the P_{i,set}), a percentage of the peak pressure (e.g., 5%, 10%, 15%, 20%, etc. of the Pₚₑₐₖ), or other determination or calculation (e.g., a pressure bound, P_{bound}, described with respect to operation 404 in FIG. 4). If the inspiratory pressure difference (ΔPᵢ) between the target inspiratory pressure (Pᵢ, calculated at operation 310) and the initial inspiratory pressure (P_{i,set}, obtained at operation 304) exceeds a maximum inspiratory pressure difference (ΔP_{i,max}), then the target inspiratory pressure (Pᵢ) calculated at operation 310 may be adjusted such that the inspiratory pressure difference does not exceed the maximum inspiratory pressure difference. For example, if the initial inspiratory pressure (P_{i,set}) is 40 cmH₂O, the target inspiratory pressure (Pᵢ) is calculated to be 20 cmH₂O, and the maximum inspiratory pressure difference (ΔP_{i,max}) is 10 cmH₂O, then the target inspiratory pressure (Pᵢ) may be adjusted to 30 cmH₂O at operation 310.

One or more target breaths may be delivered at operation 312 based on the target rise time constant (τ) and the target inspiratory pressure (Pᵢ) calculated at operation 310. The target rise time constant (τ) and/or the target inspiratory pressure (Pᵢ) may then be adjusted or updated during ventilation of the patient based on performance operations 308-322, as described herein.

Operations 308-312 may be repeated as required or desired. For example, the respiratory parameters determined at operation 308 may be continuously or periodically re-determined. For instance, the respiratory parameters may be determined after a threshold number of consecutive breaths have been delivered (e.g., 10 breaths, 20 breaths, 30 breaths, 100 breaths, etc.) or after a certain period of time has passed (e.g., one minute, five minutes, thirty minutes, one hour, five hours, ten hours, 24 hours, etc.) or in response to a change in patient status (such as health status, physiological parameters, surgery, or other changes). A change in one or more of the respiratory parameters may result in a change in the target rise time constant (τ) and/or the target inspiratory pressure (Pᵢ) calculated at operation 310.

At operation 314, an actual tidal volume (V_{T}) is determined for delivered breaths. The actual tidal volume (V_{T}) is determined for the target breath(s) delivered at operation 312. The actual tidal volume (V_{T}) may be calculated based on measured pressure and/or flow characteristics of gases delivered during the target breath(s). The actual tidal volume for the inspiratory phase of the target breath may be measured using a flow sensor of a ventilator. In contrast to the target tidal volume (V_{T, set}) set at operation 302, which is a tidal volume desired to be delivered to a patient during an inspiratory phase of a breath, the actual tidal volume (V_{T}) is the tidal volume actually delivered to the patient during an inspiratory phase of a breath. The actual tidal volume (V_{T}) is affected by the target rise time constant (τ) and/or the target inspiratory pressure (PO.

At operation 316, the actual tidal volume (V_{T}) is compared with the initial ventilation settings. In particular, the actual tidal volume (V_{T}) may be compared with the target tidal volume (V_{T,set}). Comparison of the actual tidal volume (V_{T}) with the target tidal volume (V_{T,set}) may be used to evaluate how well the values calculated for the target rise time constant (τ) and/or the target inspiratory pressure (Pᵢ) are achieving the desired tidal volume. In an ideal situation, the actual tidal volume (V_{T}) would be the same (or substantially the same) as the targeted tidal volume (V_{T,set}). However, due to the complexities of the ventilation system and patient physiology, that is not always the case. The comparison in operation 316 may thus be used in operation 318, below, when determining whether to make an adjustment to the target rise time constant (τ) and/or the target inspiratory pressure (Pᵢ) (e.g., to cause the actual tidal volume to be closer to the target tidal volume, such as the actual tidal volume falling within a threshold range, or volume range, of the target tidal volume).

At operation 318, a determination is made as to whether to adjust the target inspiratory pressure. In an example, if the actual tidal volume (V_{T}) differs from the target tidal volume (V_{T,set}) by more than a threshold amount, the determination is made to change the target inspiratory pressure and the method proceeds “YES” to operation 320. At operation 320, the target inspiratory pressure (Pᵢ) is adjusted. Alternatively, if the actual tidal volume does not differ from the target tidal volume by more than the threshold amount, the determination is made not to adjust the target inspiratory pressure, and the method proceeds “NO” to operation 312, where a breath is delivered according to the existing target rise time constant (τ) and the target inspiratory pressure (Pᵢ), without changing the target rise time constant (τ) or the target inspiratory pressure (Pᵢ). One example of determining whether to adjust the target inspiratory pressure (Pᵢ), and making the corresponding adjustments, is described below with respect to FIG. 4.

At operation 322, a target breath is delivered, based on the target rise time constant (τ) calculated at operation 310 and the adjusted target inspiratory pressure (Pᵢ) adjusted at operation 320. The method 300 may then proceed back to operation 314 where an actual tidal volume (V_{T}) is measured for determining if further adjustment of the target inspiratory pressure (Pᵢ) is required or desired.

FIG. 4 is a flowchart illustrating another example method 400 for lung-protective ventilation. Specifically, FIG. 4 describes operations 402-406 that further describe adjustment of the target inspiratory pressure (PO, as discussed at determination 318 and operation 320 in FIG. 3.

At determination 402, it is determined whether the actual tidal volume (V_{T}) (e.g., measured at operation 314 in FIG. 3) is within a threshold range. The threshold range is based on the target tidal volume (V_{T,set}), which may be set by a clinician or otherwise calculated based on patient information (e.g., as described at operation 302 in FIG. 3). For example, the threshold range may be the target tidal volume (V_{T,set}) plus or minus a volume error (ΔV_{T}). In an instance, the volume error may be a percentage of the target tidal volume (V_{T,set}), such as 1%, 3%, 5%, 10%, etc. In another instance, the volume error may be a specified volume, such as 0.1 mL, 0.5 mL, 0.7 mL, 1 mL, etc. When the actual tidal volume (V_{T}) is determined to fall within the threshold range (V_{T,set}±ΔV_{T}), the actual tidal volume (V_{T}) may be determined to be substantially the same as the set tidal volume (V_{T,set}). As a result of this determination, the target inspiratory pressure (Pᵢ) and/or the target rise time constant (τ) are maintained as calculated at operation 310, and the method proceeds “YES” to operation 312 in FIG. 3.

If, alternatively, the method determines (at determination operation 402) that the actual tidal volume is outside the threshold range, then the target inspiratory pressure (Pᵢ) and/or the target rise time constant (τ) may be required or desired to be adjusted. In this situation, the method proceeds “NO” to operation 404.

At operation 404, a pressure bound is calculated. In an example, the pressure bound is based on the target inspiratory pressure calculated for the prior breath (e.g., the target inspiratory pressure calculated at operation 310 in FIG. 3) and a difference between the actual tidal volume (V_{T}) and the target tidal volume (V_{T,set}). In an example, the pressure bound may be determined using Eqn. 6, below:

$${P}_{b o u n d} ( k ) = {P}_{i} ( k - 1 ) + Gain \times \frac{{V}_{T , set} - {V}_{T}}{{c}_{R S}} , 0 \text{.5} \leq Gain \leq 0.9 \qquad ( 6 )$$

where, Pᵢ(k−1) is the target inspiratory pressure for the last delivered breath, C_{RS} is the dynamically estimated lung compliance (e.g., as determined at operation 308 in FIG. 3), and Gain is a constrained value used to control the adjustment of the inspiratory pressure from breath-to-breath. In an example, the Gain is set to a default value of 0.75.

An increase in the target inspiratory pressure (Pᵢ) and/or a decrease in the target rise time (τ) may increase the actual tidal volume delivered to the patient. For example, higher flow is delivered to obtain a higher pressure and flow is ramped up faster to obtain higher target rise times. Alternatively, a decrease in the target inspiratory pressure (Pᵢ) and/or an increase in the target rise time (τ) may decrease the actual tidal volume delivered to the patient. For example, lower flow is delivered to obtain a lower pressure and flow is ramped up slower to obtain lower target rise times. Thus, when determining a pressure bound based on the difference in actual tidal volume (V_{T}) and the target tidal volume (V_{T,set}), the pressure bound may be greater than or less than the target inspiratory pressure (Pᵢ) calculated for the prior breath.

If the target tidal volume is greater than the actual tidal volume, such that a difference between target tidal volume and actual tidal volume is positive (e.g., V_{T,set}>V_{T}), then the pressure bound is greater than the target inspiratory pressure (e.g., P_{bound}>Pᵢ). If the target tidal volume is less than the actual tidal volume, such that a difference between target tidal volume and actual tidal volume is negative (e.g., V_{T,set}<V_{T}), then the pressure bound is less than the target inspiratory pressure (e.g., P_{bound}<Pᵢ).

At operation 406, the target inspiratory pressure (Pᵢ) is adjusted to the pressure bound (P_{bound}). If the target tidal volume (V_{T,set}) is greater than the actual tidal volume (V_{T}) (e.g., less tidal volume is delivered than desired, V_{T,set}>V_{T}), then the target inspiratory pressure (Pᵢ) is increased to the pressure bound (P_{bound}). If the target tidal volume (V_{T,set}) is less than the actual tidal volume (V_{T}) (e.g., more tidal volume is delivered than desired, V_{T,set}<V_{T}), then the target inspiratory pressure (Pᵢ) is decreased to the pressure bound (P_{bound}). Operation 406 may be similar to operation 320 in FIG. 3. For example, the adjusted target inspiratory pressure in operation 320 may be the pressure bound calculated at operation 404. Flow then proceeds to operation 322 in FIG. 3.

By having the ventilator determine the inspiratory time constant and/or update the inspiratory time constant dynamically during ventilation as described herein, the likelihood of lung injury may be reduced by preventing incorrectly set inspiratory time constants. In addition, as the patient's condition changes, the target pressure and/or the inspiratory time constant may also be dynamically updated during ventilation to provide improved ventilation that results in full tidal volume delivery at lower mechanical power and peak flows.

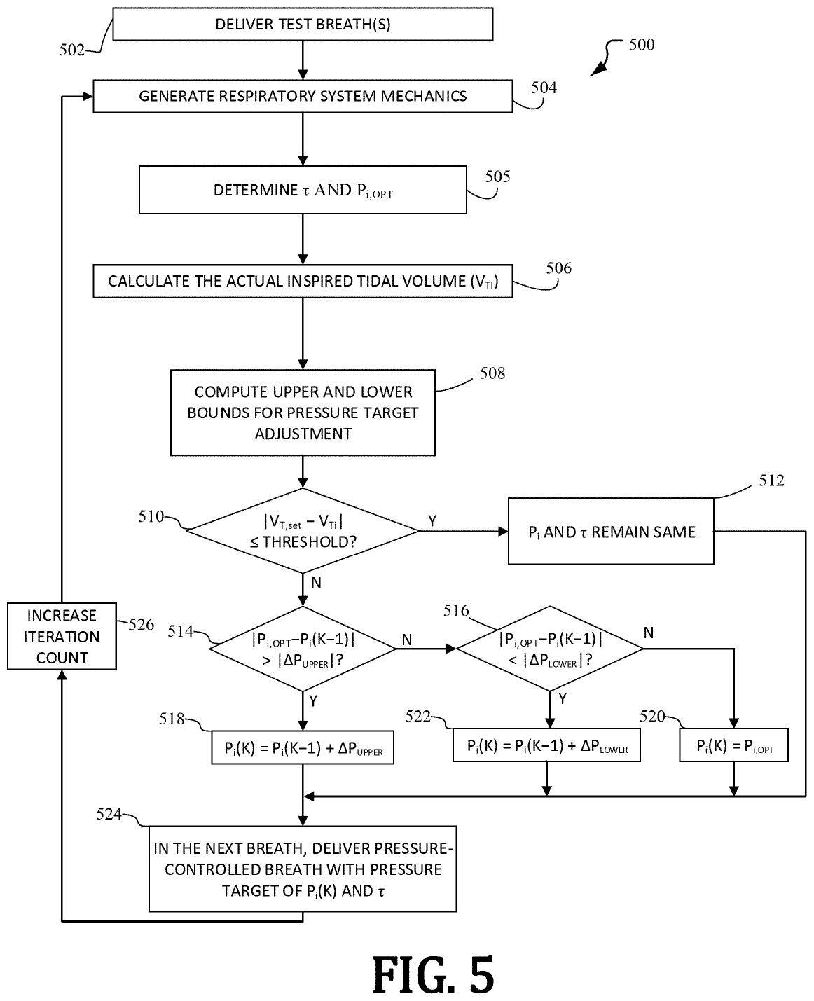

FIG. 5 is a flowchart illustrating another example method 500 for lung-protective ventilation. At operation 502, one or more test breaths are delivered to a patient that is connected to the ventilator. The test breath(s) are delivered according to initial parameters and a target tidal volume (V_{T,set}). For instance, an initial target pressure (Pᵢ(0)) may be set to 10 cmH₂O and an initial target rise time constant (τ(0)) may be set to one-third of the inspiratory time (e.g., Tᵢ/3). Other initial parameters are also possible.

At operation 504, respiratory system mechanics are generated or determined by the ventilator. In the initialization state, the respiratory system mechanics are generated based on the one or more test breaths delivered in operation 502, and the respiratory system mechanics may be generated at the end of one or more of the inhalation phases of the test breaths. The respiratory system mechanics may include compliance, elastance, and/or resistance, which may be dynamic compliance and resistance, as discussed above. For example, the respiratory mechanics may include the resistance of the patient's airway (R_{aw}), the elastance of the patient's respiratory system E_{RS}, and/or the compliance of the patient's respiratory system (C_{RS}, where E_{RS}=1/C_{RS}).

At operation 505, the target rise time constant (τ) and the target inspiratory pressure (Pᵢ) are determined or calculated. The target rise time constant (τ) and/or the target inspiratory pressure (Pᵢ) may be calculated or determined in the same manner as discussed above with reference to operation 310 in method 300. The target rise time constant (τ) and the target inspiratory pressure (Pᵢ) determined in operation 505 may also be referred to as the optimal target rise time constant (τₒₚₜ) and the optimal target inspiratory pressure (P_{i,opt}). In the first iteration of method 500, operation 506 may also include delivering a target breath based on the target rise time constant (τ) and the target inspiratory pressure (Pᵢ).

At operation 506, the actual inspired tidal volume (V_{Ti}) of the delivered target breath for the current iteration is calculated. At operation 508, upper and/or lower bounds for pressure target adjustment are computed. As one example, the lower pressure bound (ΔP_{lower}) may be equal to 0.5×(V_{T,set}−Vₜᵢ)/C_{DYN} and the upper pressure bound (ΔPᵤₚₚₑᵣ) may be equal to 0.9×(V_{T,set}−Vₜᵢ)/C_{DYN}.

At operation 510, an absolute value of the difference between the actual inspired tidal volume (V_{Ti}) and the set tidal volume (V_{t,set}) is compared to a tidal volume threshold. The tidal volume threshold may be 5% of the set tidal volume or 0.5 mL, or the greater of the two. Other threshold values may also be used. If the absolute value of the difference between the actual inspired tidal volume (V_{Ti}) and the set tidal volume (V_{t,set}) is less than or equal to the tidal volume threshold, the method 500 flows to operation 512 where target rise time constant (τ) and the target inspiratory pressure (Pᵢ) remain the same for the next breath.

If, at operation 510, the absolute value of the difference between the actual inspired tidal volume (V_{Ti}) and the set tidal volume (V_{t,set}) is greater than the tidal volume threshold, the method flows to operation 514. At operation 514, a determination is made as to whether the absolute value of the difference between the optimal target inspiratory pressure (P_{i,opt}), determined in operation 505, and the pressure target used for the prior breath Pᵢ(k−1)) is compared to the absolute value of the upper pressure bound (|ΔPᵤₚₚₑᵣ|). If the absolute value of the difference between the optimal target inspiratory pressure (P_{i,opt}) and the target inspiratory pressure used for the prior breath (Pᵢ(k−1)) is greater than the absolute value of the upper pressure bound (|ΔPᵤₚₚₑᵣ|) the method flows to operation 518. At operation 518, the target inspiratory pressure for the upcoming breath (Pᵢ(k)) is set to the target inspiratory pressure used for the previous breath ((Pᵢ(k−1)) plus the upper pressure bound (ΔPᵤₚₚₑᵣ).

If, at operation 514, the absolute value of the difference between the optimal target inspiratory pressure (P_{i,opt}) and the target inspiratory pressure used for the prior breath (Pᵢ(k−1)) is less than or equal to the absolute value of the upper pressure bound (|ΔPᵤₚₚₑᵣ|), the method 500 flows to operation 516. At operation 516, the absolute value of the difference between the optimal target inspiratory pressure (P_{i,opt}) and the target inspiratory pressure used for the prior breath (Pᵢ(k−1)) is compared to the absolute value of the lower pressure bound (|ΔP_{lower}|). If the absolute value of the difference between the optimal target inspiratory pressure (P_{i,opt}) and the target inspiratory pressure used for the prior breath (Pᵢ(k−1)) is less than the lower pressure bound (|ΔP_{lower}|), the method 500 flows to operation 522. At operation 522, the target inspiratory pressure for the upcoming breath (Pᵢ(k)) is set to the target inspiratory pressure used for the previous breath ((Pᵢ(k−1)) plus the lower pressure bound (ΔP_{lower}).

If, at operation 516, the absolute value of the difference between the optimal target inspiratory pressure (P_{i,opt}) and the target inspiratory pressure used for the prior breath (Pᵢ(k−1)) is greater than or equal to the absolute value of the lower pressure bound (|ΔP_{lower}|), the method 500 flows to operation 520. At operation 520, the target inspiratory pressure for the upcoming breath (Pᵢ(k)) is set to the optimal target inspiratory pressure (P_{i,opt}) determined in operation 505.

At operation 524, the next breath is delivered as a pressure-controlled breath with the pressure target (Pᵢ(k)) as set in one of operations 512, 518, 520, or 522, respectively, and the target rise time constant (τ) calculated in operation 505. The method 500 then flows to operation 526 where the iteration counter is increased for the next breath. For instance, the breath that was delivered in operation 524 becomes considered the previous breath. As an example, operation 526 may include setting Pᵢ(k−1) equal to the Pᵢ(k) that was used for delivering the breath in operation 524.

Method 500 then returns to operation 504, where operation 504 and the subsequent operations are performed for the next breath. The method 500 may repeat for each breath or for a set of breaths, such as every 5, 10, 20, or 50 breaths, among other potential numbers of breaths. The repeated operations for determining the target inspiratory pressure (Pᵢ(k)) and the target rise time constant (τ) may be performed during an exhalation phase of a breath to determine the target settings of the inhalation phase of the next breath.

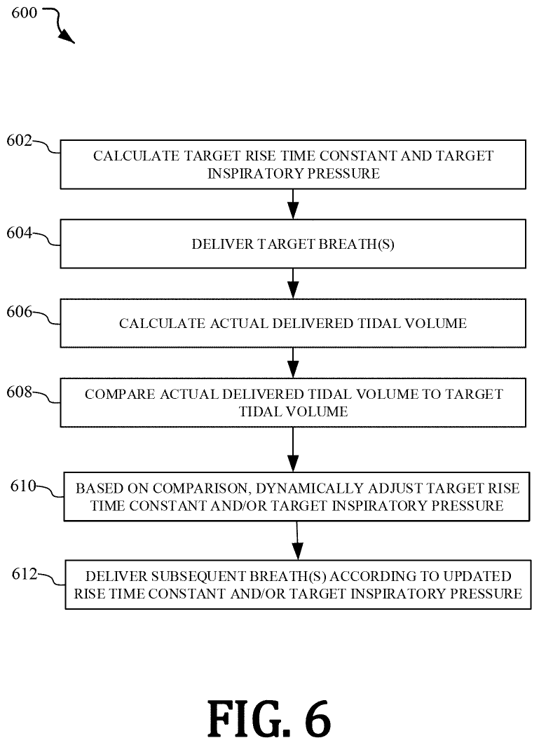

FIG. 6 is a flowchart illustrating another example method 600 for lung-protective ventilation. At operation 602, a target rise time constant and a target inspiratory pressure are calculated based on respiratory mechanics of the patient and a target tidal volume. The calculation of the target rise time constant (τ) and/or the target inspiratory pressure (Pᵢ) may be further based on a target inspiratory volume, a peak inspiratory flow, and/or a mechanical power value. The target inspiratory volume, the peak flow, and/or the mechanical power value may be input by a clinician in some examples, as discussed above.

At operation 604, one or more first target breaths are delivered to the patient according to the target rise time constant (τ) and/or the target inspiratory pressure (Pᵢ) calculated in operation 602. Calculating the target rise time constant (τ) and/or the target inspiratory pressure (Pᵢ) may be performed using any of the methods and techniques described herein. At operation 606, the actual tidal volume delivered to the patient in the first target breath(s) is calculated or determined. For example, operation 606 may include measuring pressure and/or flow values associated with the delivered breath and calculating the actual delivered tidal volume based on the measured pressure and/or flow values.

At operation 608, the actual delivered tidal volume, calculated in operation 606, is compared to the target inspiratory tidal volume. Comparing the actual delivered tidal volume to the target inspiratory tidal volume may include determining the difference between the actual delivered tidal volume and the target inspiratory tidal volume.

At operation 610, the target rise time constant and/or the target inspiratory pressure are dynamically adjusted based on the comparison performed in operation 608. For example, based on determining that the actual delivered tidal volume differs from the target inspiratory volume by greater than a threshold value, the target rise time constant and/or the target inspiratory pressure may be adjusted to compensate for the difference between the actual delivered tidal volume and the target inspiratory volume. At operation 612, one or more subsequent or second breaths are delivered to the patient according to the adjusted target rise time constant and/or the adjusted target inspiratory pressure.

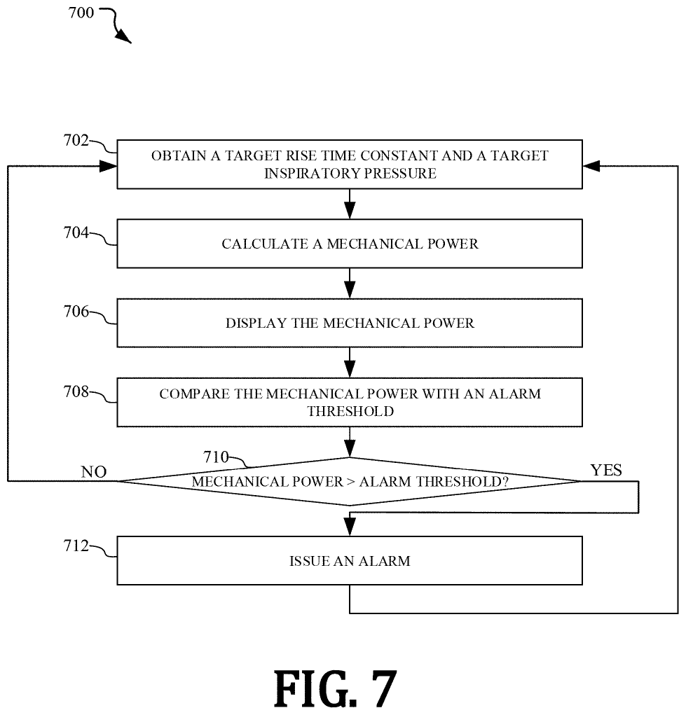

FIG. 7 is a flowchart illustrating an example method 700 for monitoring mechanical power. At operation 702, a target rise time constant (τ) and a target inspiratory pressure (Pᵢ) are obtained. As described herein, the target rise time constant (τ) and the target inspiratory pressure (Pᵢ) may be calculated and/or adjusted during ventilation. For example, the target rise time constant (τ) and the target inspiratory pressure (Pᵢ) may be obtained similarly to operations 304, 310, and/or 320 in FIG. 3 and/or operation 406 in FIG. 4.

At operation 704, the method calculates a mechanical power to be delivered to the patient according to the values obtained in operation 702. For instance, the mechanical power may be calculated based on the target rise time constant (τ) and the target inspiratory pressure (Pᵢ) obtained at operation 702. In an example, the mechanical power may be calculated based on Eqn. 5, above. Inputs to the equation other than the target rise time constant (τ) and the target inspiratory pressure (Pᵢ) may also be obtained, such as manually inputted by a clinician, preset, measured, or otherwise determined based on patient parameters (e.g., PBW, gender, height, patient type, etc.). For example, the following inputs may be obtained prior to calculating the mechanical power: a respiratory rate (f), an actual tidal volume (V_{T}), a target tidal volume (V_{T,set}), a PEEP level, an inspiratory time (Tᵢ), and/or respiratory parameters (R_{aw}, E_{RS}, C_{RS}, τ_{RS}, etc.).

At operation 706, the mechanical power (e.g., calculated at operation 704) is displayed. The mechanical power may be displayed on a display of a ventilator. For example, the mechanical power may be displayed proximate a set of outputs and/or current patient ventilation parameters. The display of the mechanical power value may also be displayed with other indicia to provide additional insights as to whether the mechanical power is low, high, and/or near an average or median value. The indicia may include a scale (e.g., ranging from low to high), colors (e.g., green, red, etc.), or other types of indicia. In some examples, the indicia may be based on an upper limit for mechanical power. For instance, the indicia may indicate the relationship of the calculated mechanical power value to a mechanical power threshold. Thus, the clinician is able to readily see if the mechanical power being delivered, or to be delivered, to the patient exceeds, or is projected to exceed, a mechanical power threshold.

At operation 708, the calculated mechanical power is compared with an alarm threshold. The alarm threshold may be set by a clinician, preset at the ventilator, or determined based on patient parameters or other clinical data. For example, an alarm threshold for an adult patient type may be higher than an alarm threshold for a neonatal patient type. In another example, an alarm threshold for a patient with a higher PBW may be higher than an alarm threshold for a patient with a lower PBW. Alarm thresholds that are preset or determined by the ventilator may be updated from time-to-time as required or desired, such as in association with ongoing studies relating to safe ranges for delivered mechanical power. The alarm threshold may be the same upper limit threshold discussed above for displaying indicia relating to mechanical power.

At determination 710, it is determined if the mechanical power is greater than the alarm threshold. Mechanical power values exceeding the alarm threshold may be associated with unsafe ventilation (e.g., the patient may be at risk of VILI). A mechanical power exceeding the alarm threshold may be an indication that the rise time constant (τ) is too short and/or the inspiratory pressure (Pᵢ) is too high for lung-protective ventilation. If the mechanical power exceeds the alarm threshold, flow proceeds “YES” to operation 712, where an alarm is issued. The alarm may include audial, visual, haptic, or any other feedback or alert. Additionally, the alarm may be associated with text or instructions displayed on a display of the ventilator associated with the calculated mechanical power, such as how much the mechanical power exceeds the alarm threshold and/or suggestions for adjusting the rise time constant (τ) and/or the inspiratory pressure (Pᵢ). If, alternatively, the mechanical power does not exceed the alarm threshold, flow proceeds “NO” back to operation 702, without issuing an alarm.

In some examples, upon the determination that the mechanical power exceeds the alarm threshold in operation 710, the ventilator may automatically adjust the ventilation settings to reduce the mechanical power, such as by changing the target rise time constant (τ) and/or a target inspiratory pressure (Pᵢ). In other examples, instead of automatically adjusting the ventilation settings, the ventilator may display suggested settings or changes to the ventilation settings to reduce the mechanical power and prompt a user to accept the suggested settings. In the lung protection ventilation modes described herein, such suggestions may be to lower the peak inspiratory flow. The suggestion may be for a ventilation setting value (e.g., peak inspiratory flow) that would reduce the mechanical power below the alarm threshold.

Operations 702-712 may repeat as required or desired. For example, each time a target rise time constant (τ) and/or a target inspiratory pressure (Pᵢ) is obtained or adjusted, a new mechanical power may be calculated. In an instance, a different mechanical power may be calculated for an initial rise time constant (τₛₑₜ) and initial inspiratory pressure (Pᵢ,set) (e.g., such as those obtained at operation 304 in FIG. 3), a target rise time constant (τ) and target inspiratory pressure (Pᵢ) (such as those calculated at operation 310 in FIG. 3), and/or adjusted target rise time constant and adjusted target inspiratory pressure (such as those determined at operation 320 in FIG. 3 and/or operation 406 in FIG. 4).

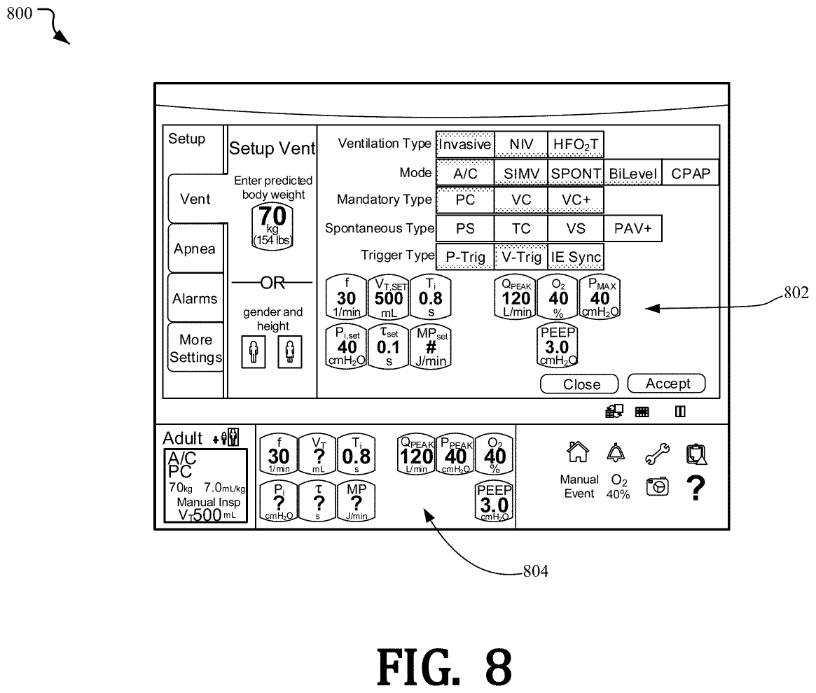

FIG. 8 shows an example graphical user interface 800 for lung-protective ventilation. The graphical user interface 800 may be displayed on a display of a ventilator. Elements of the graphical user interface 800 may include inputs 802 and outputs 804.

As shown in FIG. 8, inputs 802 may be received, determined, and/or calculated for a breath mode. The inputs 802 may include initial ventilation settings 802, such as ventilation type, mode, mandatory type or spontaneous type, trigger type, respiratory flow rate (f), target tidal volume (V_{T,set}), inspiratory time (Tᵢ), peak flow (Qₚₑₐₖ), oxygen level, peak pressure (Pₚₑₐₖ), a PEEP level, a predicted body weight, and/or gender and height of the patient. The inputs 802 may also include an initial inspiratory pressure (P_{i,set}), an initial rise time constant (τₛₑₜ), and/or an initial mechanical power or maximum mechanical power (MPₛₑₜ).

Outputs 804 may also be shown on the graphical user interface 800. The outputs 804 may include actual tidal volume (V_{T}), target inspiratory pressure (Pᵢ), target rise time constant (τ), and/or delivered mechanical power (MP). The outputs 804 may also include one or more of the inputs 802.

Although the present disclosure discusses the implementation of these techniques in the context of a ventilator capable of performing one or more breath modes, such as volume-targeted pressure-controlled ventilation, the techniques introduced above may be implemented for a variety of medical devices, or devices utilizing flow sensors, which may not have specified breath modes. A person of skill in the art will understand that the technology described in the context of a medical ventilator for human patients could be adapted for use with other systems such as ventilators for non-human patients or general gas transport systems. Additionally, a person of ordinary skill in the art will understand that the modeled exhalation flow may be implemented in a variety of breathing circuit setups.

Those skilled in the art will recognize that the methods and systems of the present disclosure may be implemented in many manners and as such are not to be limited by the foregoing aspects and examples. In other words, functional elements being performed by a single or multiple components, in various combinations of hardware and software or firmware, and individual functions, can be distributed among software applications at either the client or server level or both. In this regard, any number of the features of the different aspects described herein may be combined into single or multiple aspects, and alternate aspects having fewer than or more than all of the features herein described are possible.

Functionality may also be, in whole or in part, distributed among multiple components, in manners now known or to become known. Thus, a myriad of software/hardware/firmware combinations are possible in achieving the functions, features, interfaces and preferences described herein. Moreover, the scope of the present disclosure covers conventionally known manners for carrying out the described features and functions and interfaces, and those variations and modifications that may be made to the hardware or software firmware components described herein as would be understood by those skilled in the art now and hereafter. In addition, some aspects of the present disclosure are described above with reference to block diagrams and/or operational illustrations of systems and methods according to aspects of this disclosure. The functions, operations, and/or acts noted in the blocks may occur out of the order that is shown in any respective flowchart. For example, two blocks shown in succession may in fact be executed or performed substantially concurrently or in reverse order, depending on the functionality and implementation involved.

Further, as used herein and in the claims, the phrase “at least one of element A, element B, or element C” is intended to convey any of: element A, element B, element C, elements A and B, elements A and C, elements B and C, and elements A, B, and C. In addition, one having skill in the art will understand the degree to which terms such as “about” or “substantially” convey in light of the measurements techniques utilized herein. To the extent such terms may not be clearly defined or understood by one having skill in the art, the term “about” shall mean plus or minus ten percent.

Numerous other changes may be made which will readily suggest themselves to those skilled in the art and which are encompassed in the spirit of the disclosure and as defined in the appended claims. While various aspects have been described for purposes of this disclosure, various changes and modifications may be made which are well within the scope of the disclosure. Numerous other changes may be made which will readily suggest themselves to those skilled in the art and which are encompassed in the spirit of the disclosure and as defined in the claims.

What is claimed is:

1. A method for lung-protective ventilation, the method comprising:
receiving, as input to the ventilator, a target tidal volume and a peak inspiratory flow;
delivering an initial breath based on initial ventilation settings;
determining respiratory parameters, based on the delivered initial breath;
calculating a target rise time constant and a target inspiratory pressure, based at least on the received target tidal volume, the peak inspiratory flow, and the determined respiratory parameters; and
delivering a target breath according to the target rise time constant and target inspiratory pressure.

2. The method of claim 1, wherein the initial ventilation settings include an initial rise time constant, an initial inspiratory pressure, and a peak inspiratory pressure.

3. The method of claim 2, the method further comprising:
measuring an actual tidal volume delivered during the target breath;
adjusting the target inspiratory pressure to an adjusted inspiratory pressure, based on the actual tidal volume; and
delivering a third breath according to the target rise time and the adjusted inspiratory pressure.

4. The method of claim 3, wherein adjusting the target inspiratory pressure is further based on a difference between the actual tidal volume and the target tidal volume.

5. The method of claim 1, wherein rise time is not available as a setting capable of being adjusted by a clinician.

6. The method of claim 1, the method further comprising calculating a mechanical power value based on the target rise time constant and the target inspiratory pressure.

7. The method of claim 6, the method further comprising displaying the calculated mechanical power and indicia indicating a relationship between the calculated mechanical power value and a mechanical power threshold.

8. The method of claim 1, wherein calculating the target rise time constant and the target inspiratory pressure is based on a least squares optimization technique.

9. The method of claim 1, wherein the target breath is delivered according to a flow profile based on the target rise time constant and the target inspiratory pressure, wherein a flow of breathing gases delivered to a patient is variable based on the flow profile.

10. The method of claim 1, the method further comprising:
displaying at least one of:
the target rise time;
the target inspiratory pressure; or
a mechanical power value calculated based on the target rise time and the target inspiratory pressure.

11. A method for lung-protective ventilation, the method comprising:
receiving a target tidal volume and peak inspiratory flow as inputs to the ventilator;
determining respiratory parameters;
calculating a rise time constant and a first target inspiratory pressure based at least on the respiratory parameters and the target tidal volume;
delivering a first breath according to the rise time constant and the first target inspiratory pressure;
measuring an actual tidal volume delivered during the first breath;
determining that the actual tidal volume is outside of a volume range from the target tidal volume;
adjusting the first target inspiratory pressure to a second target inspiratory pressure; and
delivering a second breath, based on the rise time constant and the second target inspiratory pressure.

12. The method of claim 11, wherein the actual tidal volume is below the volume range, and the second target inspiratory pressure is greater than the first target inspiratory pressure.

13. The method of claim 11, the actual tidal volume is higher than the volume range, and the second target inspiratory pressure is lower than the first target inspiratory pressure.

14. The method of claim 11, wherein calculating the rise time constant and the first target inspiratory pressure is further based on a peak inspiratory pressure.

15. The method of claim 11, the method further comprising:
receiving an initial rise time constant and an initial inspiratory pressure; and
delivering an initial breath, based on the initial rise time constant and the initial inspiratory pressure, wherein respiratory parameters are based on the initial breath.

16. A ventilator for lung-protective ventilation, the ventilator comprising:
a flow valve;
a flow sensor;
a user interface;
a processor; and
memory storing instructions that, when executed by the processor, cause the ventilator to perform a set of operations comprising:
receiving, at the user interface, a target tidal volume and a peak inspiratory flow;
delivering a first breath through the flow valve;
determining respiratory parameters, based on the first breath;
calculating a target rise time constant and a first target inspiratory pressure, based at least on the determined respiratory parameters, the target tidal volume, and the peak inspiratory flow;
delivering a second breath, through the flow valve according to the target rise time constant and the first target inspiratory pressure;
calculating, based on measurements from the flow sensor, an actual tidal volume delivered during the second breath;
adjusting the first target inspiratory pressure to a second target inspiratory pressure, based on a difference between the actual tidal volume and the target tidal volume; and
delivering a third breath, through the flow valve according to the target rise time and the second target inspiratory pressure.

17. The ventilator of claim 16, wherein the operations further comprise:
displaying, in the user interface, the actual tidal volume and at least one of: the target rise time constant, the first target inspiratory pressure, or the second target inspiratory pressure.

18. The ventilator of claim 16, wherein the first breath and the second breath are delivered according to a volume-targeted, pressure-controlled, mandatory breath mode.

19. The ventilator of claim 16, further comprising:
a pressure sensor, wherein the flow valve is controlled based at least on a measurement from the pressure sensor while delivering the second breath and the third breath.

20. The ventilator of claim 16, wherein the set of operations further comprise:
calculating a mechanical power for the third breath, based at least on the target rise time and the second target inspiratory pressure; and
alarming based on the calculated mechanical power.
